(19) United States
(12) Patent Application Publication
(10) Pub. No.: US 2012/0298981 A1
(43) Pub. Date: Nov. 29, 2012

(54) STYRENE-BASED COPOLYMERS, IN PARTICULAR FOR USE IN OPTOELECTRONIC COMPONENTS

(75) Inventors: Niels Schulte, Kelkheim (DE); Aurélie Ludemann, Frankfurt Am Main (DE); Junyou Pan, Frankfurt Am Main (DE); René Peter Scheurich, Gross-Zimmern (DE); Thomas Eberle, Landau (DE); Anna Hayer, Mainz (DE); Philipp Stoessel, Frankfurt Am Main (DE); Rémi Manouk Anémian, Seoul (KR)

(73) Assignee: MERCK PATENT GMBH, Darmstadt (DE)

(21) Appl. No.: 13/575,856

(22) PCT Filed: Dec. 23, 2010
(86) PCT No.: PCT/EP2010/007912

§ 371 Date: Jul. 27, 2012

(30) Foreign Application Priority Data
Jan. 29, 2010 (DE) 10 2010 006 377.0

(51) Int. Cl. C08F 212/32; G02B 5/22; H01L 51/54; C07C 211/61; H01L 51/30; H01L 51/46; H01B 1/12; C07C 15/60

(52) U.S. Cl. 257/40; 526/280; 252/500; 252/582; 585/26; 564/434; 257/E51.025; 257/E51.026; 257/E51.027

(57) ABSTRACT

The present invention relates to styrene-based copolymers having recurring units which contain substituted anthra [symbol] cenes in the side chain, to blends comprising these polymers according to the invention, and to the use of these polymers and blends in electronic devices. The invention furthermore relates to electronic devices which contain these polymers or blends.

The present invention relates to styrene-based copolymers having recurring units which contain substituted anthracenes in the side chain, to blends comprising these polymers according to the invention, and to the use of these polymers and blends in electronic devices. The invention furthermore relates to electronic devices which contain these polymers or blends.

Organic electronic devices, for example opto-electronic devices, which are based on organic materials deposited from the gas phase are distinguished by very good technical properties, such as, for example, high stability, a long lifetime and a low operating voltage. The usual procedure here is for small molecules to be applied by vapour deposition in a vacuum chamber. The term “small molecule OLED” (SMOLED) is usually used here. A “small molecule OLED” (SMOLED) consists, for example, of one or more organic hole-injection layers, hole-transport layers, emission layers, electron-transport layers and electron-injection layers as well as an anode and a cathode, where the entire system is usually located on a glass substrate. However, the vapour-deposition process has the disadvantage of the requisite use of high-performance high-vacuum techniques, which are required for the deposition of the organic materials on the substrates. This process is therefore complex and thus very expensive. In addition, not all compounds can be evaporated without decomposition.

It has therefore already been attempted to combine the very good opto-electronic properties of volatile molecules with the simple processability of polymeric systems. Such combinations are described, for example, in U.S. Pat. No. 7,250,226 B2, US 2007/0187673 A1 and U.S. Pat. No. 6,899,963. In these specifications, an aliphatic main polymer chain is generally used, where the functional units are arranged in the side chain.

Further specifications which describe this subject are, for example, JP 2005/108556, JP 2005/108552, JP 2003/346277, JP 2004/303483, JP 2004/303488, JP 2005/285661, JP 2001/257076, JP 2003/338375, JP 2004/111228, JP 2004/014325, JP 2004/303490, JP 2005/285466 and JP 2004/303489. The main polymer chain here is not involved in charge transport or emission, but instead the functionalities, such as, for example, charge transport or emission units, are arranged in the side chains.

Although all compounds described in these specifications exhibit good behaviour with respect to their processability from solution, they exhibit deficits, however, with respect to the film-formation properties and the emission colour of these compounds. In particular, deep-blue emission (CIE y coordinates in the range from 0.05 to 0.15) is desired, which cannot be achieved with the compounds known from the prior art. The lifetime is also usually inadequate, and the requisite operating voltage of the systems known from the prior art is too high. For high-quality applications, it is therefore necessary to provide emitter systems which have an improved emission colour, high stability and good film-formation properties and at the same time require only a low operating voltage.

The object of the present invention therefore consisted in the provision of such compounds.

It has been observed, entirely surprisingly, that polystyrene-based copolymers containing recurring units of the general formula (I) have, unexpectedly, deep-blue colour coordinates and high stability in addition to good film-formation properties and low operating voltages.

The present invention thus relates to polymers which contain one or more substituted and/or unsubstituted styrene recurring units and one or more recurring units of the general formula (I)

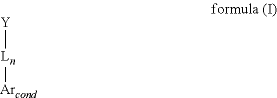

where the symbols and indices used have the following meanings:
Y denotes a link to the polymer backbone;
Lₙ is a substituted or unsubstituted aromatic, heteroaromatic or non-aromatic group, or an alkylene, alkenylene or alkynylene group, where 3≧n≧1;
Ar_{cond} denotes a condensed, aromatic ring system having 10 to 40, preferably 10 to 24, C atoms or condensed, heteroaromatic ring system having 10 to 40, preferably 10 to 24, ring atoms, where at least one ring atom is a heteroatom, preferably selected from N, O and/or S, and the other atoms are C atoms, which may be unsubstituted or substituted by one or more radicals R;
R here is in each case, independently of one another, H, D, F, Cl, Br, I, N(R¹⁰)₂, N(Ar)₂, CR¹⁰═CR¹⁰Ar, Si(R¹⁰)₃, B(OR¹⁰)₂, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which may be substituted by one or more radicals R¹⁰, where one or more non-adjacent CH₂ groups may be replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NR¹⁰, O, S or CONR¹⁰ and where one or more H atoms may be replaced by F, Cl, Br, I, CN or NO₂, or an aryl or heteroaryl group having 5 to 60 ring atoms, which may in each case be substituted by one or more radicals R¹⁰, or an aromatic or heteroaromatic ring system having 5 to 40 aromatic ring atoms, which may in each case be substituted by one or more radicals R¹⁰, or an aryloxy or heteroaryloxy group having 5 to 40 aromatic ring atoms, which may be substituted by one or more radicals R¹⁰, or a combination of these systems; where two or more substituents R may also form a mono- or polycyclic, aliphatic or aromatic ring system with one another, where at least one R is K—(Ar)ₘ, where m is greater than or equal to 1;
R¹⁰ is in each case, independently of one another, H, D or an aliphatic or aromatic hydrocarbon radical having 1 to 20 C atoms;
K is on each occurrence, in each case independently of one another, a covalent bond, a substituted or unsubstituted aromatic, heteroaromatic or non-aromatic group, or an alkylene, alkenylene or alkynylene group; and
Ar on each occurrence, independently of one another, denotes a substituted or unsubstituted aryl group, aryloxy group heteroaryl group, heteroaryloxy group, an aromatic or heteroaromatic ring system or a non-aromatic group.

Ar_{cond} is preferably a naphthyl, anthracenyl, phenanthrenyl, benzanthracenyl or pyrenyl group, which may be unsubstituted or substituted by one or more radicals R.

The linking to Lₙ in the formula (I) preferably takes place via the 1- or 2-position in the case of the naphthyl group, preferably takes place via the 2-, 6- or 9-position in the case of the anthracenyl group, preferably takes place via the 2- or 3-position in the case of the phenanthrenyl group, preferably takes place via the 2- or 12-position in the case of the benzanthracenyl group and preferably takes place via the 1-, 2- or 3-position in the case of the pyrenyl group.

Ar_{cond} is particularly preferably an anthracenyl group, which is preferably linked to Lₙ in the 2-, 6- or 9-position, particularly preferably in the 9-position.

Particular preference is thus given to polymers which contain one or more substituted and/or unsubstituted styrene recurring units and one or more recurring units of the general formula (Ia)

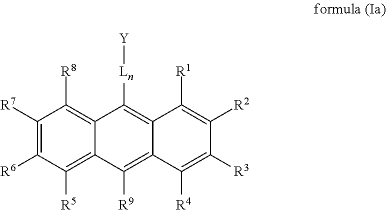

where the symbols and indices used have the following meanings:
Y denotes a link to the polymer backbone;
Lₙ is a substituted or unsubstituted aromatic, heteroaromatic or non-aromatic group, or an alkylene, alkenylene or alkynylene group, where 3≧n≧1;
R¹ to R⁹ are each, independently of one another, H, D, F, Cl, Br, I, N(R¹⁰)₂, N(Ar)₂, CR¹⁰═CR¹⁰Ar, Si(R¹⁰)₃, B(OR¹⁰)₂, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which may be substituted by one or more radicals R¹⁰, where one or more non-adjacent CH₂ groups may be replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NR¹⁰, O, S or CONR¹⁰ and where one or more H atoms may be replaced by F, Cl, Br, I, CN or NO₂, or an aryl or heteroaryl group having 5 to 60 ring atoms, which may in each case be substituted by one or more radicals R¹⁰, or an aromatic or heteroaromatic ring system having 5 to 40 aromatic ring atoms, which may in each case be substituted by one or more radicals R¹⁰, or an aryloxy or heteroaryloxy group having 5 to 40 aromatic ring atoms, which may be substituted by one or more radicals R¹⁰, or a combination of these systems; where two or more substituents R¹ to R⁹ may also form a mono- or polycyclic, aliphatic or aromatic ring system with one another, where at least one representative from R¹ to R⁹ is K—(Ar)ₘ, where m is greater than or equal to 1;
R¹⁰ is in each case, independently of one another, H, D or an aliphatic or aromatic hydrocarbon radical having 1 to 20 C atoms;
K is on each occurrence, in each case independently of one another, a covalent bond, a substituted or unsubstituted aromatic, heteroaromatic or non-aromatic group, or an alkylene, alkenylene or alkynylene group; and
Ar on each occurrence, independently of one another, denotes a substituted or unsubstituted aryl group, aryloxy group heteroaryl group, heteroaryloxy group, an aromatic or heteroaromatic ring system or a non-aromatic group.
   m is preferably selected so that it corresponds to the maximum number of possible substitution positions on K. If, for example, K is a single covalent bond, m=1. If, by contrast, K is a phenyl, m=1, 2, 3, 4 or at most 5.

An aryl group or aryloxy group in the sense of the present invention preferably contains 5 to 60 C atoms; a heteroaryl group or heteroaryloxy group in the sense of the present invention contains 2 to 60 C atoms and at least one heteroatom, with the proviso that the sum of C atoms and heteroatoms is at least 5. The heteroatoms are preferably selected from Si, N, P, O, S and/or Se. An aryl group or heteroaryl group here is taken to mean either a simple aromatic ring, i.e. benzene, or a simple heteroaromatic ring, for example pyridine, pyrimidine, thiophene, or a condensed aryl or heteroaryl group, for example naphthalene, anthracene, phenanthrene, quinoline, isoquinoline, benzothiophene, benzofuran and indole.

An aromatic ring system in the sense of the present invention contains 5 to 60 C atoms in the ring system. A heteroaromatic ring system in the sense of the present invention contains 2 to 60 C atoms and at least one heteroatom in the ring system, with the proviso that the sum of C atoms and heteroatoms is at least 5. The heteroatoms are preferably selected from Si, N, P, O, S and/or Se. An aromatic or heteroaromatic ring system in the sense of the present invention is, in addition, intended to be taken to mean a system which does not necessarily contain only aryl or heteroaryl groups, but instead in which, in addition, a plurality of aryl or heteroaryl groups may be interrupted by a non-aromatic unit (preferably less than 10% of the atoms other than H), such as, for example, an sp³-hybridised C or N or O atom. Thus, for example, systems such as 9,9′-spirobifluorene, 9,9-diarylfluorene, triarylamine, diaryl ether and stilbene are also intended to be taken to be aromatic ring systems in the sense of the present invention, as are systems in which two or more aryl groups are interrupted, for example, by a linear or cyclic alkyl group, a silyl group, benzophenones, phosphine oxides and sulfoxides.

An aromatic or heteroaromatic ring system having 5 to 60 ring atoms, which may in each case also be substituted by any desired radicals, preferably the radicals defined under R¹⁰, and which may be linked to the aromatic or heteroaromatic ring system via any desired positions, is taken to mean, in particular, groups derived from benzene, naphthalene, anthracene, phenanthrene, pyrene, chrysene, perylene, fluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine, benzothiadiazole, benzanthrene, benzanthracene, rubicene and triphenylene.

A non-aromatic group in the sense of the present invention is a group which does not have a cyclically conjugated system having (4n+2) π-electrons, for example an alkyl group, alkenyl group, alkynyl group or alkoxy group or, if the non-aromatic group has two bonding partners, correspondingly an alkylene, alkenylene, alkynylene or an alkoxylene group.

For the purposes of the present invention, an alkyl group having 1 to 40 C atoms, in which, in addition, individual H atoms or CH₂ groups may be substituted by the above-mentioned groups, is preferably taken to mean the radicals methyl, ethyl, n-propyl, i-propyl, cyclopropyl, n-butyl, i-butyl, s-butyl, t-butyl, cyclobutyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl and octynyl.

An alkoxy group having 1 to 40 C atoms is preferably taken to mean methoxy, trifluoromethoxy, ethoxy, n-propoxy, i-propoxy, n-butoxy, i-butoxy, s-butoxy, t-butoxy and 2-methylbutoxy.

Alkylene, alkenylene and alkynylene groups which are preferred in accordance with the invention are those having 2 to 40 C atoms, where, in addition, one or more H atoms or CH₂ groups in the alkylenes or one or more H atoms or HC═CH groups in the alkenylenes or one or more C≡C groups in the alkynylenes may be replaced by the above-mentioned groups. Preference is given to the radicals methylene, ethylene, n-propylene, i-propylene, cyclopropylene, n-butylene, i-butylene, s-butylene, t-butylene, cyclobutylene, 2-methylbutylene, n-pentylene, s-pentylene, cyclopentylene, n-hexylene, cyclohexylene, n-heptylene, cycloheptylene, n-octylene, cyclooctylene, 2-ethylhexylene, trifluoromethylene, pentafluoroethylene, 2,2,2-trifluoroethylene, ethenylene, propenylene, butenylene, pentenylene, cyclopentenylene, hexenylene, cyclohexenylene, heptenylene, cycloheptenylene, octenylene, cyclooctenylene, ethynylene, propynylene, butynylene, pentynylene, hexynylene and octynylene.

In a further embodiment of the present invention, the polymer is characterised by the general formula (II),

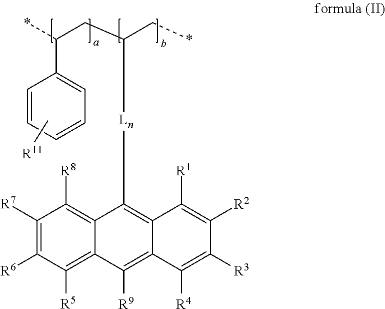

where the symbols and indices have the meaning indicated above and
R¹¹ is equal to H, D, F, Cl, Br, I, N(Ar)₂, CR¹²═CR¹²Ar, Si(R¹²)₃, B(OR¹²)₂, a straight-chain alkyl, alkoxy or thioalkoxy group having 1 to 40 C atoms or a branched or cyclic alkyl, alkoxy or thioalkoxy group having 3 to 40 C atoms, each of which may be substituted by one or more radicals R¹², where one or more non-adjacent CH₂ groups may be replaced by R¹²C═CR¹², C≡C, Si(R¹²)₂, Ge(R¹²)₂, Sn(R¹²)₂, C═O, C═S, C═Se, C═NR¹², P(═O)(R¹²), SO, SO₂, NR¹², O, S or CONR¹² and where one or more H atoms may be replaced by F, Cl, Br, I, CN or NO₂, or an aryl or heteroaryl group having 5 to 40 ring atoms, which may in each case be substituted by one or more radicals R¹², or an aromatic or heteroaromatic ring system having 5 to 40 aromatic ring atoms, which may in each case be substituted by one or more radicals R¹², or an aryloxy or heteroaryloxy group having 5 to 40 aromatic ring atoms, which may be substituted by one or more radicals
R¹², or a combination of these systems; where two or more radicals R¹² may form a mono- or polycyclic, aliphatic or aromatic ring system;
R¹² is on each occurrence, in each case independently of one another, H or an aliphatic or aromatic hydrocarbon radical having 1 to 20 C atoms;
a is a styrene-based recurring unit and b is a recurring unit of the general formula (I).

The proportion of the recurring unit of the general formula (I) in the polymer according to the invention is preferably in the range from 0.01 to 99.99 mol %, particularly preferably in the range from 10 to 90 mol % and in particular in the range from 25 to 75 mol %, based on the entire polymer. Correspondingly, the proportion of the styrene unit in the polymer according to the invention is preferably 99.99 to 0.01 mol %, particularly preferably 90 to 10 mol %, and in particular 75 to 25 mol %, based on the entire polymer.

The number-average molecular weight Mₙ of the polymer according to the invention is preferably in the range 2000 to 2,000,000 g/mol, particularly preferably in the range from 3000 to 1,500,000 g/mol, and in particular in the range from 5000 to 250,000 g/mol. The number average molecular weight Mₙ is determined by GPC (gel permeation chromatography) using an internal polystyrene standard.

The polymer according to the invention preferably has an aliphatic chain as backbone. This is preferably obtained by polymerisation of compounds of the general formula (I) which, as monomers, contain corresponding polymerisable groups (see compound of the general formula (III), described below) with styrene or a styrene derivative containing polymerisable groups. The polymerisable groups are preferably vinyl, vinyl ester, vinyl ether, vinylamide, acrylate, methacrylate and acrylamide. The polymerisable group used may likewise be groups which can be converted into a polymer by cationic, anionic or ring-opening polymerisation. It is likewise possible to employ combinations of the said polymerisable groups.

For the purposes of the present invention, it is furthermore preferred for one or more of the radicals R¹ to R⁹ and/or R¹¹ (R¹, R², R³, R⁴, R⁵, R⁶, R⁷, R⁸, R⁹ and/or R¹¹) in the compound of the general formula (I) or (II) each, independently of one another, to denote an electron-transport group, an electron-injection group, an electron-blocking group, a hole-transport group, a hole-injection group, a hole-blocking group, a photon-absorption group, an exciton-generating group and/or an emitter group.

A hole-injection group and/or hole-transport group in the sense of the present invention is a group having an energetically high HOMO (“highest occupied molecular orbital”), preferably >−5.8 eV, particularly preferably >−5.5 eV. This supports hole injection.

The hole-injection and/or hole-transport group is preferably a triarylamine, benzidine, tetraaryl-para-phenylenediamine, triarylphosphine, phenothiazine, phenoxazine, dihydrophenazine, thianthrene, dibenzo-para-dioxin, phenoxathiin, carbazole, azulene, thiophene, pyrrole and furan derivative and in addition an O-, S-, Se- or N-containing heterocycle having a high HOMO (HOMO=highest occupied molecular orbital). These arylamines and heterocycles preferably result in an HOMO in the polymer of greater than −5.8 eV (vs. vacuum level), particularly preferably greater than −5.5 eV.

An electron-injection and/or electron-transport group in the sense of the present invention is a group having a low LUMO (LUMO=lowest unoccupied molecular orbital), preferably <−1.5 eV, particularly preferably <−2.0 eV (vs. vacuum level). This supports electron injection.

The electron-injection and/or electron-transport group is preferably a pyridine, pyrimidine, pyridazine, pyrazine, oxadiazole, quinoline, quinoxaline, anthracene, benzanthracene, pyrene, perylene, benzimidazole, triazine, ketone, phosphine oxide and phenazine derivative, but also triarylboranes and further O-, S-, Se or N-containing heterocycles having a low LUMO can be used. These units preferably result in an LUMO in the polymer of less than −1.5 eV (vs. vacuum level), particularly preferably less than −2.0 eV.

Possible for the purposes of the present invention is a combination of hole-injection group and/or hole-transport group and electron-injection and/or electron-transport group, where these simultaneously have a high HOMO and a low LUMO.

A photon-absorption group in the sense of the present invention is preferably a group which is capable of absorbing a photon of any desired energy or any desired wavelength, preferably in the spectral region of visible light. It is generally a dye. Suitable dyes are, for example, those which are usually also used in organic photovoltaic cells, in dye-sensitised solar cells, in charge-generation layers or in xerographic devices. Preferred dyes are, for example, perylenes and derivatives thereof (Angew. Chem. Int. Ed. 2006, 45, 3364-3368), ruthenium dyes and derivatives thereof (Nature, 1991, 353, p. 737 and Angew. Chemie. Int. Ed. 2005, 44, 5740-5744), phtalocyanines, azo dyes, rylenes, perylenediimides, perylenebisdicarboximides, terrylenes, quaterrylenes, phorphyrins, squarines and derivatives thereof.

An exciton-generating group in the sense of the present invention is preferably taken to mean a group which is capable of generating an exciton by recombination of a hole and an electron.

An emitter group is a group which is capable of emitting light, for example a fluorescent or phosphorescent dye. Fluorescent dyes are predominantly singlet emitters. A triplet emitter group in the sense of the present invention is preferably a group which is also able to emit light from the triplet state at room temperature with high efficiency, i.e. exhibits electrophosphorescence instead of electrofluorescence, which frequently causes an increase in the energy efficiency. Suitable for this purpose are firstly compounds which contain heavy atoms having an atomic number of greater than 36. Preference is given to compounds which contain d or f transition metals which meet the above-mentioned condition. Particular preference is given here to corresponding structural units which contain elements from group 8 to 10 (Ru, Os, Rh, Ir, Pd, Pt). Suitable structural units for the polymers according to the invention here are, for example, various complexes, as described, for example, in WO 02/068435 A1, WO 02/081488 A1, EP 1239526 A2 and WO 2004/026886 A2. Corresponding monomers are described in WO 02/068435 A1 and in WO 2005/042548 A1.

In addition, functional groups which improve the transfer from the singlet state to the triplet state and which, employed in support of the emitter groups, improve the phosphorescence properties of these structural elements may be present in the polymer according to the invention. Suitable for this purpose are, in particular, carbazole and bridged carbazole dimer units, as described, for example, in WO 2004/070772 A2 and WO 2004/113468 A1. Also suitable for this purpose are ketones, phosphine oxides, sulfoxides, sulfones, silane derivatives and similar compounds, as described, for example, in WO 2005/040302 A1.

Suitable further emitter groups in the sense of the present invention are aromatic structures having 6 to 40 C atoms or also tolan, stilbene or bisstyrylarylene derivatives, each of which may be substituted by one or more radicals R. Particular preference is given here to the incorporation of 1,4-phenylene, 1,4-naphthylene, 1,4- or 9,10-anthrylene, 1,6-, 2,7- or 4,9-pyrenylene, 3,9- or 3,10-perylenylene, 4,4′-biphenylylene, 4,4″-terphenylylene, 4,4′-bi-1,1′-naphthylylene, 4,4′-tolanylene, 4,4′-stilbenzylene, 4,4″-bisstyrylarylene, benzothiadiazole and corresponding oxygen derivatives, quinoxaline, phenothiazine, phenoxazine, dihydrophenazine, bis(thiophenyl)arylene, oligo(thiophenylene), phenazine, rubrene, pentacene or perylene derivatives, which are preferably substituted, or preferably conjugated push-pull systems (systems which are substituted by donor and acceptor substituents) or systems such as squarines or quinacridones, which are preferably substituted.

According to an embodiment of the present invention, R² and/or R⁹ is preferably an aromatic or heteroaromatic group having 6 to 20 ring atoms.

According to a further embodiment of the present invention, R² and/or R⁷ is preferably a charge-transport group.

Particular preference is given to polymers in which the recurring unit of the general formula (I) is formed by the following monomers (1) to (6):

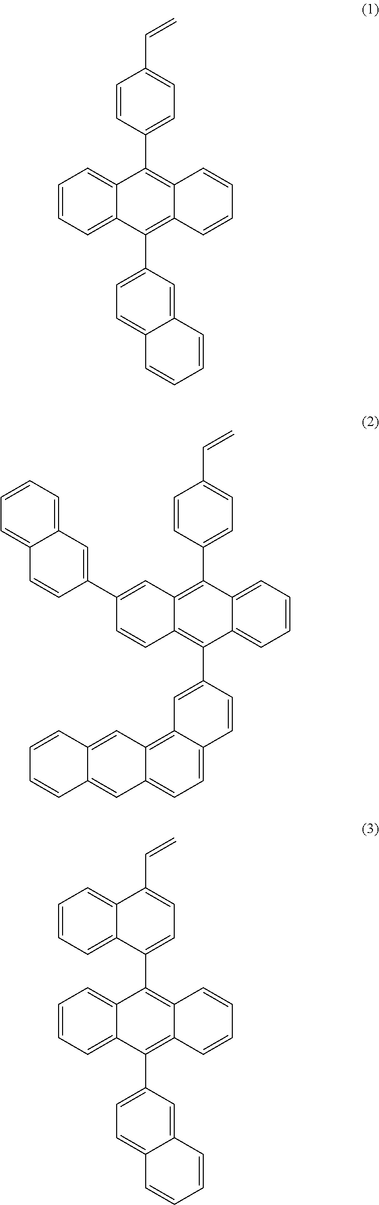

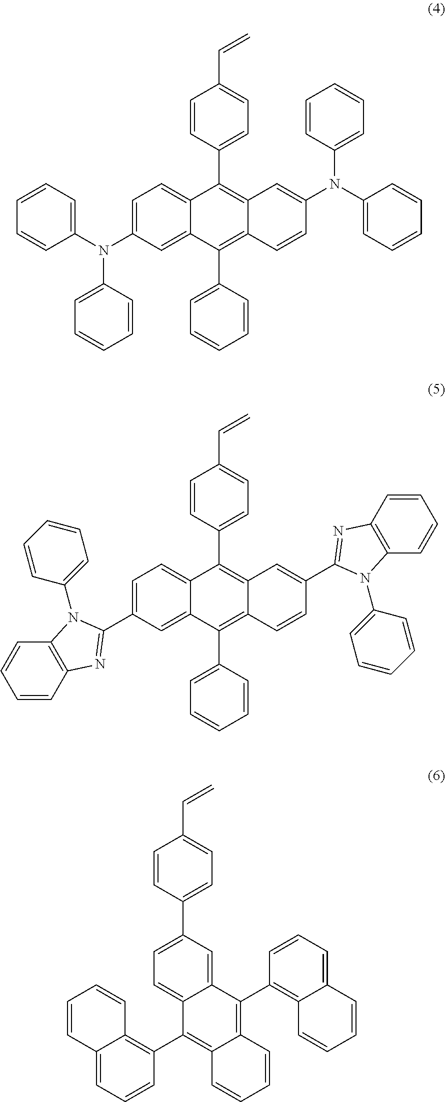

Particularly preferred polymers are consequently copolymers of the formulae (IIa), (IIb), (IIc), (IId), (IIe) and (IIf), as shown below:

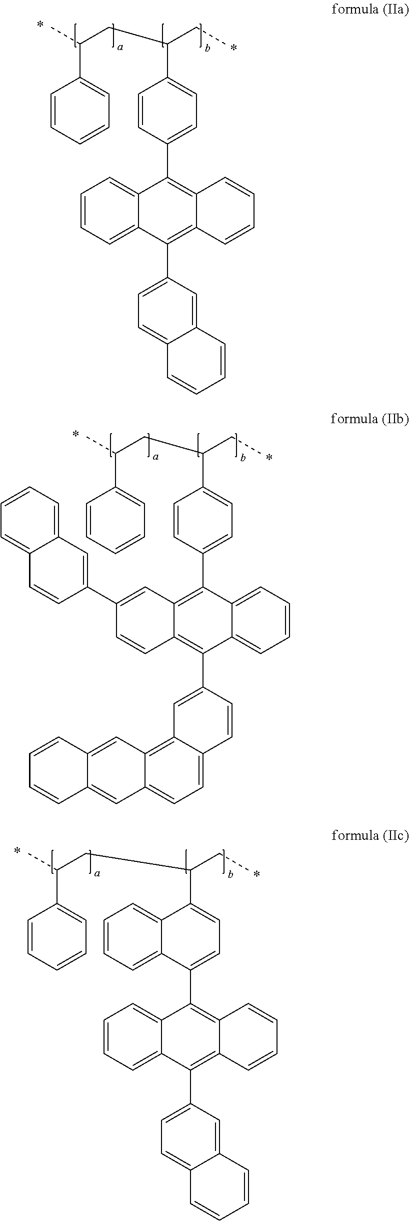

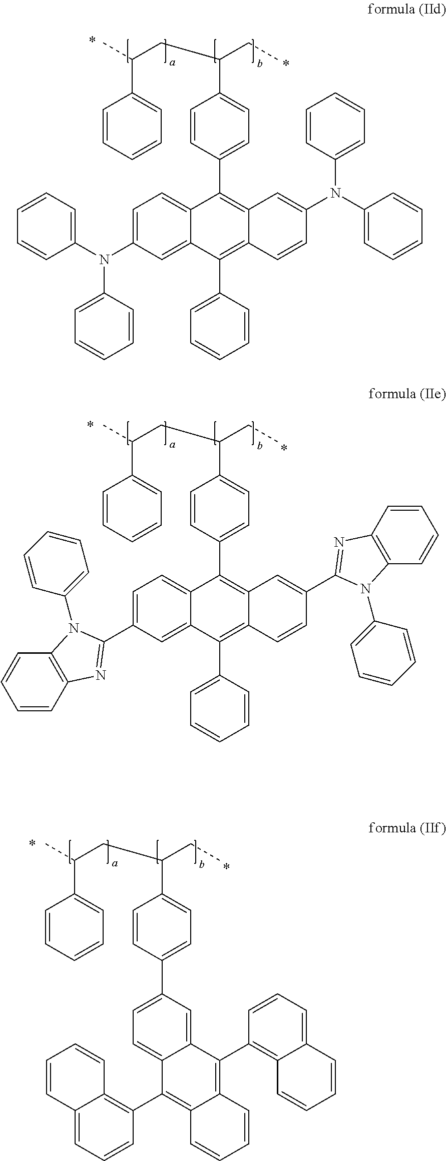

In addition, it may be preferred to use the polymers according to the invention not as the pure substance, but instead in the form of a mixture (blend) together with further polymeric, oligomeric, dendritic or low-molecular-weight substances of any desired type. These may improve, for example, the electronic properties, themselves emit or function as matrix material.

The term “mixture” or “blend” above and below denotes a mixture comprising at least one polymeric component according to the invention.

In an embodiment of the present invention, the polymers according to the invention are preferably employed as emitting compounds in an emitting layer. An organic electroluminescent device here may comprise one emitting layer or it may comprise a plurality of emitting layers, where at least one emitting layer comprises at least one polymer according to the invention, as defined above. If a plurality of emission layers are present, these preferably have in total a plurality of emission maxima between 380 nm and 750 nm, resulting overall in white emission, i.e. various emitting compounds which are able to fluoresce or phosphoresce are used in the emitting layers. Particular preference is given to three-layer systems, where the three layers exhibit blue, green and orange or red emission (for the basic structure see, for example, WO 05/011013).

If the polymers according to the invention are employed as emitting compounds in an emitting layer, they are preferably employed in combination with one or more matrix materials. The mixture of the polymers according to the invention and the at least one matrix material comprises between 1 and 99% by weight, preferably between 10 and 98% by weight and particularly preferably between 30 and 97% by weight, of the matrix material, based on the mixture as a whole comprising emitter polymer and matrix material. Correspondingly, the mixture comprises between 1 and 99% by weight, preferably between 2 and 90% by weight and particularly preferably between 3 and 70% by weight, of the polymers according to the invention, based on the mixture as a whole comprising emitter polymer and matrix material.

Preferred matrix materials are CBP (N,N-biscarbazolylbiphenyl), carbazole derivatives (for example in accordance with WO 05/039246, US 2005/0069729, JP 2004/288381), azacarbazoles (for example in accordance with EP 1617710, EP 1617711, EP 1731584, JP 2005/347160), ketones (for example in accordance with WO 04/093207), phosphine oxides, sulfoxides and sulfones (for example in accordance with WO 05/003253), oligophenylenes, aromatic amines (for example in accordance with US 2005/0069729), bipolar matrix materials (for example in accordance with WO 07/137,725) or silanes (for example in accordance with WO 05/111172).

If the polymers according to the invention are employed as matrix materials in an emitting layer, they are preferably employed in combination with one or more emitter compounds. The mixture of the polymers according to the invention and the at least one emitter compound comprises between 1 and 99% by weight, preferably between 2 and 90% by weight, particularly preferably between 3 and 40% by weight, and in particular between 5 and 15% by weight, of at least one emitter compound, based on the mixture as a whole comprising emitter compound and matrix material. Correspondingly, the mixture comprises between 99 and 1% by weight, preferably between 98 and 10% by weight, particularly preferably between 97 and 60% by weight, and in particular between 95 and 85% by weight, of the polymers according to the invention, based on the mixture as a whole comprising emitter compound and matrix material.

For the purposes of the present invention, the emitter compound in the composition according to the invention is preferably a singlet emitter, a triplet emitter or a singlet exciton-generating group, particularly preferably a singlet emitter. The singlet emitter is preferably a blue-emitting singlet emitter. The singlet emitter may likewise be a green or red singlet emitter.

Preferred singlet emitters are selected from the class of the monostyrylamines, the distyrylamines, the tristyrylamines, the tetrastyrylamines, the styrylphosphines, the styryl ethers and the arylamines.

A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. Corresponding phosphines and ethers are defined analogously to the amines. An arylamine or an aromatic amine in the sense of the present invention is taken to mean a compound which contains three substituted or unsubstituted aromatic or heteroaromatic ring systems bonded directly to the nitrogen. At least one of these aromatic or heteroaromatic ring systems is preferably a condensed ring system, preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 2,6- or 9,10-position. Aromatic pyrenamines, pyrenediamines, chrysenamines and chrysenediamines are defined analogously thereto, where the diarylamino groups on the pyrene are preferably bonded in the 1-position or in the 1,6-position.

Further preferred singlet emitters are selected from indenofluorenamines or indenofluorenediamines, for example in accordance with WO 06/122630, benzoindenofluorenamines or benzoindenofluorenediamines, for example in accordance with WO 08/006,449, and dibenzoindenofluorenamines or dibenzoindenofluorenediamines, for example in accordance with WO 07/140,847.

Examples of singlet emitters from the class of the styrylamines are substituted or unsubstituted tristilbenamines or the emitters described in WO 06/000388, WO 06/058737, WO 06/000389, WO 07/065549 and WO 07/115,610. Distyrylbenzene and distyrylbiphenyl derivatives are described in U.S. Pat. No. 5,121,029. Further styrylamines are found in US 2007/0122656 A1.

Particularly preferred styrylamine emitters are:

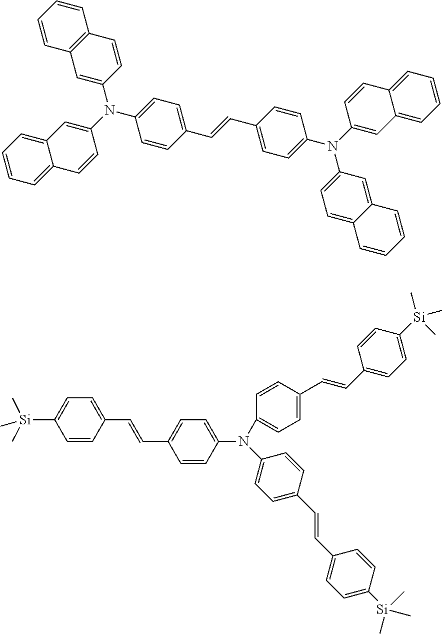

Particularly preferred triarylamine emitters are:

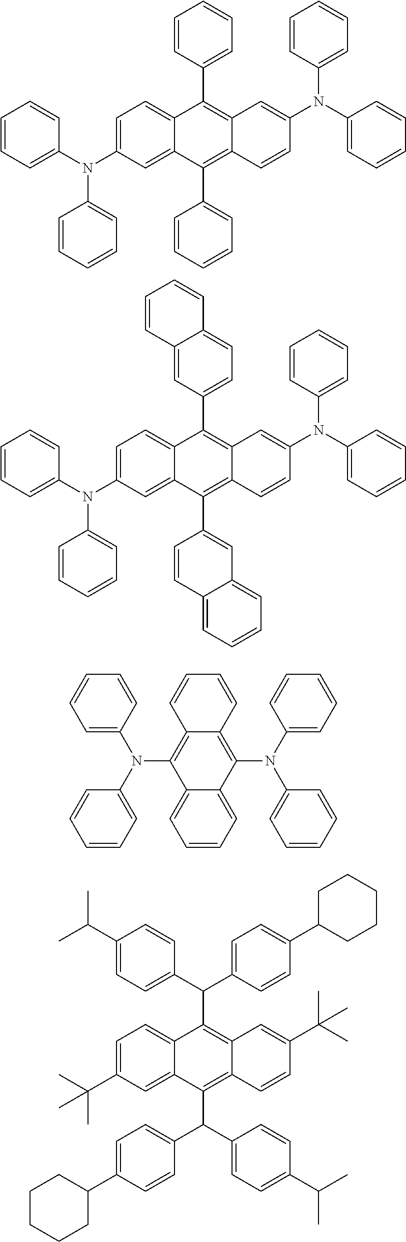

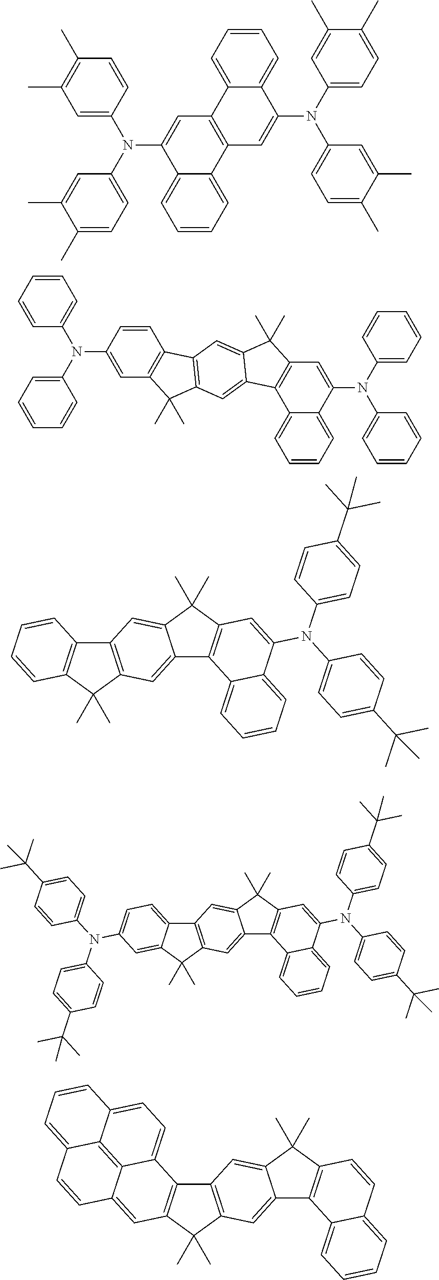

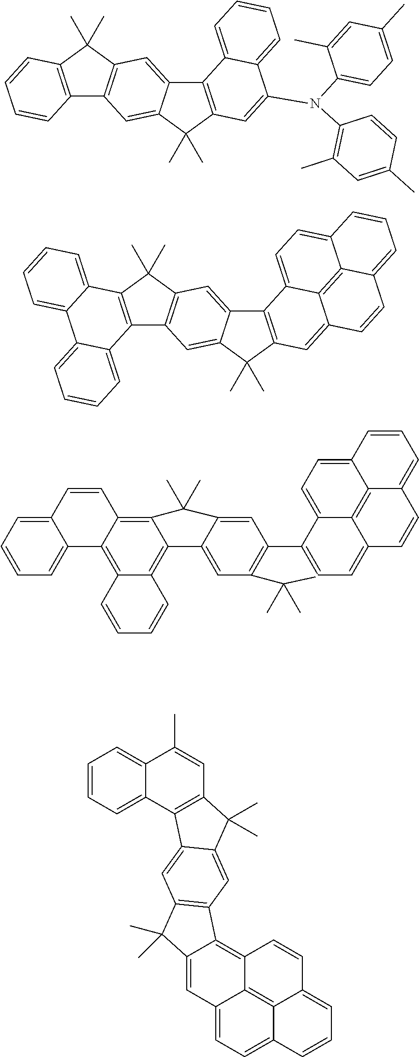

Further preferred emitters are selected from derivatives of naphthalene, anthracene, tetracene, benzanthracene, benzophenanthrene (DE 10 2009 005746), fluorene, fluoranthene, periflanthene, indenoperylene, phenanthrene, perylene (US 2007/0252517 A1), pyrene, chrysene, decacyclene, coronene, tetraphenylcyclopentadiene, pentaphenylcyclopentadiene, fluorene, spirofluorene, rubrene, coumarin (U.S. Pat. No. 4,769,292, U.S. Pat. No. 6,020,078, US 2007/0252517 A1), pyran, oxazole, benzoxazole, benzothiazole, benzimidazole, pyrazine, cinnamic acid esters, diketopyrrolopyrrole, acridone and quinacridone (US 2007/0252517 A1).

Of the anthracene compounds, particular preference is given to 9,10-substituted anthracenes, such as, for example, 9,10-diphenylanthracene and 9,10-bis(phenylethynyl)anthracene. 1,4-Bis(9′-ethynylanthracenyl)benzene is also a preferred dopant. Preference is likewise given to derivatives of rubrene, coumarin, rhodamine, quinacridone, such as, for example, DMQA (═N,N′-dimethylquinacridone), dicyanomethylenepyran, such as, for example, DCM (=4-(dicyanoethylene)-6-(4-dimethylaminostyryl-2-methyl)-4H-pyran), thiopyran, polymethine, pyrylium and thiapyrylium salts, periflanthene and indenoperylene.

Blue fluorescent emitters are preferably polyaromatic compounds, such as, for example, 9,10-di(2-naphthylanthracene) and other anthracene derivatives, derivatives of tetracene, xanthene, perylene, such as, for example, 2,5,8,11-tetra-t-butylperylene, phenylene, for example 4, 4′-(bis(9-ethyl-3-carbazovinylene)-1,1′-biphenyl, fluorene, fluoranthene, arylpyrenes (U.S. Ser. No. 11/097,352 filed Apr. 4, 2005), arylenevinylenes (U.S. Pat. No. 5,121,029, U.S. Pat. No. 5,130,603), bis(azinyl)imine-boron compounds (US 2007/0092753 A1), bis(azinyl)methene compounds and carbostyryl compounds.

Further preferred blue fluorescent emitters are described in C. H. Chen et al.: “Recent developments in organic electroluminescent materials” Macromol. Symp. 125, (1997) 1-48 and “Recent progress of molecular organic electroluminescent materials and devices” Mat. Sci. and Eng. R, 39 (2002), 143-222.

Further preferred blue-fluorescent emitters are the hydrocarbons disclosed in DE 10 2008 035413.

Suitable phosphorescent compounds (triplet emitters) are, in particular, compounds which emit light, preferably in the visible region, on suitable excitation and in addition contain at least one atom having an atomic number greater than 38 and less than 84, particularly preferably greater than 56 and less than 80.

Examples of the emitters described above are revealed by WO 00/70655, WO 01/41512, WO 02/02714, WO 02/15645, EP 1191613, EP 1191612, EP 1191614, WO 05/033244 and DE 10 2008 015526. In general, all phosphorescent complexes as used in accordance with the prior art for phosphorescent OLEDs and as are known to the person skilled in the art in the area of organic electroluminescence are suitable, and the person skilled in the art will be able to use further phosphorescent complexes without inventive step.

In a further embodiment according to the invention, the triplet emitter preferably contains an organometallic compound unit. The organometallic compound unit is preferably an organometallic coordination compound. An organometallic coordination compound is taken to mean a compound containing a metal atom or ion in the centre of the compound surrounded by an organic compound as ligand. An organometallic coordination compound is additionally characterised in that a carbon atom of the ligand is bonded to the central metal via a coordination bond.

The triplet emitter compound is preferably a metal complex comprising a metal selected from the group consisting of the transition metals, the rare earths, the lanthanoids and the actinoids, preferably Ir, Ru, Os, Eu, Au, Pt, Cu, Zn, Mo, W, Rh, Pd and Ag, particularly preferably Ir.

It is furthermore preferred for the organic ligand to be a chelate ligand. A chelate ligand is taken to mean a bi- or polydentate ligand, which is able to bond to the central metal correspondingly via two or more atoms.

The polymers according to the invention can be dissolved in one or more solvents. The present invention thus furthermore relates to solutions and formulations comprising one or more polymers or blends according to the invention in one or more solvents. The way in which solutions of this type can be prepared is known to the person skilled in the art and is described, for example, in WO 02/072714 A1, WO 03/019694 A2 and the literature cited therein. Suitable and preferred solvents for formulations are, for example, toluene, anisole, xylene, methyl benzoate, dimethylanisole, mesitylene, tetralin, veratrol and tetrahydrofuran, and mixtures thereof.

These solutions can be used in order to produce thin polymer layers, for example by surface-coating methods (for example spin coating) or by printing processes (for example ink-jet printing).

Preference is also given in accordance with the invention to polymers containing structural units of the formula (I) which additionally contain one or more polymerisable, and thus crosslinkable, groups. These are particularly suitable for the production of films or coatings, in particular for the production of structured coatings, for example by thermal or light-induced in-situ polymerisation and in-situ crosslinking, such as, for example, in-situ UV photopolymerisation or photopatterning. Particular preference is given for such applications to polymers according to the invention containing one or more additional polymerisable groups selected from acrylate, methacrylate, vinyl, epoxy and oxetane. It is possible here to use both corresponding polymers as pure substances, but it is also possible to use formulations or blends of these polymers as described above. These can be used with or without addition of solvents and/or binders. Suitable materials, processes and devices for the processes described above are disclosed, for example, in WO 2005/083812 A2. Possible binders are, for example, polystyrene, polycarbonate, polyacrylate, polyvinylbutyral and similar, opto-electronically neutral polymers. Preference is furthermore given to polymers according to the invention containing fluorine-containing groups.

In a deposited polymer layer, such groups generate a layer which, in a similar way to a crosslinked polymer, cannot be detached again due to F—F interactions. This offers advantages on application of further layers from solution.

The polymer according to the invention or the further polymeric, oligomeric or dendrimeric compounds in the blend may contain additional structural units which are different from the above-mentioned structural units and originate, for example, from the following classes:
group 1: units which influence the hole-injection and/or hole-transport properties of the polymers;
group 2: units which influence the electron-injection and/or electron-transport properties of the polymers;
group 3: units which have combinations of individual units from group 1 and group 2;
group 4: units which modify the emission characteristics to such an extent that electrophosphorescence can be obtained instead of electrofluorescence;
group 5: units which improve transfer from the so-called singlet state to the triplet state;
group 6: units which influence the emission colour of the resultant polymers;
group 7: units which are typically used as backbone;
group 8: units which influence the morphological/film-formation properties and/or the rheological properties of the resultant polymers.

The structural units can be in the form of individual molecules, compounds or in the form of oligomers or polymers. Preferred polymers or compounds are those in which at least one structural unit has charge-transport properties, i.e. which comprise units from group 1 and/or 2.

Structural units from group 1 which have hole-injection and/or hole-transport properties are, for example, triarylamine, benzidine, tetraaryl-para-phenylenediamine, triarylphosphine, phenothiazine, phenoxazine, dihydrophenazine, thianthrene, dibenzo-para-dioxin, phenoxathiyne, carbazole, azulene, thiophene, pyrrole and furan derivatives and further O-, S-, Se- or N-containing heterocycles having a high-lying HOMO (HOMO=highest occupied molecular orbital). These arylamines and heterocycles preferably result in an HOMO in the polymer of greater than −5.8 eV (against vacuum level), particularly preferably greater than −5.5 eV.

Structural units from group 2 which have electron-injection and/or electron-transport properties are, for example, pyridine, pyrimidine, pyridazine, pyrazine, oxadiazole, quinoline, quinoxaline, anthracene, benzanthracene, pyrene, perylene, benzimidazole, triazine, ketone, phosphine oxide and phenazine derivatives, but also triarylboranes and further O-, S- or N-containing heterocycles having a low-lying LUMO (LUMO=lowest unoccupied molecular orbital). These units in the polymer preferably result in an LUMO of less than −1.5 eV (against vacuum level), particularly preferably less than −2.0 eV.

It may be preferred for the polymers to comprise units from group 3 in which structures which influence the hole mobility and structures which increase the electron mobility (i.e. units from groups 1 and 2) are bonded directly to one another or structures which influence both the hole mobility and the electron mobility. Some of these units can serve as emitters and shift the emission colour into the green, yellow or red. Their use is thus suitable, for example, for the generation of other emission colours from originally blue-emitting polymers.

Structural units from group 4 are those which are able to emit light from the triplet state with high efficiency, even at room temperature, i.e. exhibit electrophosphorescence instead of electrofluorescence, which frequently causes an increase in the energy efficiency. Suitable for this purpose are firstly compounds which contain heavy atoms having an atomic number of greater than 36. Preference is given to compounds which contain d- or f-transition metals which satisfy the above-mentioned condition. Particular preference is given here to corresponding structural units which contain elements from groups 8 to 10 (Ru, Os, Rh, Ir, Pd, Pt). Suitable structural units for the polymers according to the invention here are, for example, various complexes, as described, for example, in WO 02/068435 A1, WO 02/081488 A1, EP 1239526 A2 and WO 2004/026886 A2. Corresponding monomers are described in WO 02/068435 A1 and in WO 2005/042548 A1.

Structural units from group 5 are those which improve transfer from the singlet state to the triplet state and which, employed in support of the structural units from group 4, improve the phosphorescence properties of these structural units. Suitable for this purpose are, in particular, carbazole and bridged carbazole dimer units, as described, for example, in WO 2004/070772 A2 and WO 2004/113468 A1. Also suitable for this purpose are ketones, phosphine oxides, sulfoxides, sulfones, silane derivatives and similar compounds, as described, for example, in WO 2005/040302 A1.

Structural units from group 6, besides those mentioned above, are those which have at least one further aromatic structure or another conjugated structure which does not fall under the above-mentioned groups, i.e. which have only little influence on the charge-carrier mobilities, are not organometallic complexes or do not influence singlet-triplet transfer. Structural elements of this type can influence the emission colour of the resultant polymers. Depending on the unit, they can therefore also be employed as emitters. Preference is given here to aromatic structures having 6 to 40 C atoms and also tolan, stilbene or bisstyrylarylene derivatives, each of which may be substituted by one or more radicals R. Particular preference is given here to the incorporation of 1,4-phenylene, 1,4-naphthylene, 1,4- or 9,10-anthrylene, 1,6-, 2,7- or 4,9-pyrenylene, 3,9- or 3,10-perylenylene, 4,4′-biphenylylene, 4,4″-terphenylylene, 4,4′-bi-1,1′-naphthylylene, 4,4′-tolanylene, 4,4′-stilbenzylene, 4,4″-bisstyrylarylene, benzothiadiazole and corresponding oxygen derivatives, quinoxaline, phenothiazine, phenoxazine, dihydrophenazine, bis(thiophenyl)arylene, oligo(thiophenylene), phenazine, rubrene, pentacene or perylene derivatives, which are preferably substituted, or preferably conjugated push-pull systems (systems which are substituted by donor and acceptor substituents) or systems such as squarines or quinacridones, which are preferably substituted.

Structural units from group 7 are units which contain aromatic structures having 6 to 40 C atoms, which are typically used as polymer backbone. These are, for example, 4,5-dihydropyrene derivatives, 4,5,9,10-tetrahydropyrene derivatives, fluorene derivatives, 9,9′-spirobifluorene derivatives, phenanthrene derivatives, 9,10-dihydrophenanthrene derivatives, 5,7-dihydrodibenzoxepine derivatives and cis- and trans-indenofluorene derivatives.

Structural units from group 8 are those which influence the morphological/film-formation properties and/or the rheological properties of the polymers, such as, for example, siloxanes, long alkyl chains or fluorinated groups, but also particularly rigid or flexible units, such as, for example, liquid crystal-forming units or crosslinkable groups.

Preference is given to polymers which contain one or more units selected from groups 1 to 8. It may likewise be preferred for more than one structural unit from a group to be present at the same time.

It is likewise preferred for the polymers to comprise units which improve charge transport or charge injection, i.e. units from group 1 and/or 2; a proportion of 0.5 to 30 mol % of these units is particularly preferred; a proportion of 1 to 10 mol % of these units is very particularly preferred.

It is furthermore particularly preferred for the polymers to comprise structural units from group 7 and units from group 1 and/or 2, in particular at least 50 mol % of units from group 7 and 0.5 to 30 mol % of units from group 1 and/or 2.

The synthesis of the units from groups 1 to 8 described above and the further emitting units is known to the person skilled in the art and is described in the literature, for example in WO 2005/014689 A2, WO 2005/030827 A1 and WO 2005/030828 A1.

The invention furthermore relates to a compound of the general formula (III)

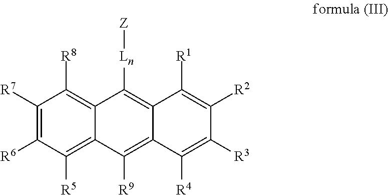

where the symbols and indices used have the following meanings:
Lₙ is a substituted or unsubstituted aromatic, heteroaromatic or non-aromatic group, or an alkylene, alkenylene or alkynylene group, where 3≧n≧1;
R¹ to R⁹ are each, independently of one another, H, D, F, Cl, Br, I, N(R¹⁰)₂, N(Ar)₂, C(═O)Ar, P(═O)Ar₂, S(═O)Ar, S(═O)₂Ar, CR¹⁰═CR¹⁰Ar, CN, NO₂, Si(R¹⁰)₃, B(OR¹⁰)₂, OSO₂R¹⁰, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which may be substituted by one or more radicals R¹⁰, where one or more non-adjacent CH₂ groups may be replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NR¹⁰, O, S or CONR¹⁰ and where one or more H atoms may be replaced by F, Cl, Br, I, CN or NO₂, or an aryl or heteroaryl group having 5 to 60 ring atoms, which may in each case be substituted by one or more radicals R¹⁰, or an aromatic or heteroaromatic ring system having 5 to 40 aromatic ring atoms, which may in each case be substituted by one or more radicals R¹⁰, or an aryloxy or heteroaryloxy group having 5 to 40 aromatic ring atoms, which may be substituted by one or more radicals R¹⁰, or a combination of these systems; where two or more substituents R¹ to R⁹ may also form a mono- or polycyclic, aliphatic or aromatic ring system with one another, where at least one representative from R¹ to R⁹ is K—(Ar)ₘ, where m is greater than or equal to 1;
R¹⁰ is in each case, independently of one another, H, D or an aliphatic or aromatic hydrocarbon radical having 1 to 20 C atoms;
K is on each occurrence, in each case independently of one another, a covalent bond, a substituted or unsubstituted aromatic, heteroaromatic or non-aromatic group, or an alkylene, alkenylene or alkynylene group;
Ar on each occurrence, independently of one another, denotes a substituted or unsubstituted aryl group, aryloxy group heteroaryl group, heteroaryloxy group, an aromatic or heteroaromatic ring system or a non-aromatic group;
Z is a polymerisable group.

The invention furthermore relates to a monomer composition comprising a substituted or unsubstituted styrene and a compound of the general formula (III)

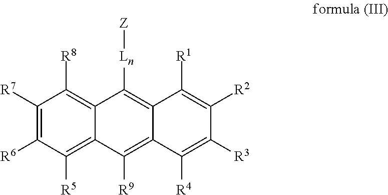

where the symbols and indices used have the meanings indicated above in relation to formula (III).

Z is preferably selected from the group consisting of oxetane, epoxide, vinyl, vinyl ether, vinyl ester, vinylamide, acrylate, methacrylate, acrylamide and methacrylamide. The polymerisable group may likewise be one which is suitable for cationic, anionic or ring-opening polymerisation.

In the monomer or monomer composition, one or more of the radicals R¹ to R⁹ in the general formula (III) can be, in each case independently of one another, an electron-transport group, an electron-injection group, an electron-blocking group, a hole-transport group, a hole-injection group, a hole-blocking group, a photon-absorption group, an exciton-generating group and/or an emitter group.

In a further embodiment, it is preferred for the monomer according to the invention or the monomer composition according to the invention to comprise one or more solvents. This is a liquid formulation which is suitable for polymerisation or copolymerisation. The present invention likewise relates to such a formulation. Suitable and preferred solvents for the formulation are preferably aprotic solvents, for example toluene, xylene, dimethyl ether or tetrahydrofuran.

The proportion of the monomer or monomer composition in the solvent or solvent mixture is preferably 0.1 to 90% by weight, particularly preferably 1 to 80% by weight, and in particular 2 to 70% by weight, based on the composition as a whole.

The monomer or monomer composition may furthermore comprise further assistants, such as, for example, stabilisers, substances which support film formation, sensitisers and the like.

The monomer according to the invention or the monomer composition according to the invention can be used for the preparation of a polymer. The polymer is preferably prepared by cationic, anionic, free-radical, ring-opening or coordinative polymerisation.

The polymer according to the invention or a blend according to the invention may in turn be dissolved in a solvent or solvent mixture, giving a formulation (see above) which is suitable for the production of electronic devices.

The formulation may furthermore comprise further components, such as, for example, further functional components (charge-transport or charge-injection units, emitter units or the like) and components which improve film formation, which serve for improving charge-carrier injection or transport or for blocking individual charge carriers. The further functional components can be, for example, those in the above-mentioned structural units from groups 1 to 8.

The polymer according to the invention or the blend exhibit excellent film-formation properties after application to a substrate from solution. In addition, the polymer or blend has excellent deep-blue colour coordinates.

The polymer is preferably applied from solution, where the polymer is correspondingly present in the electronic device as a layer after removal of the solvent or solvent mixture. The layer here can be a hole-transport layer, a hole-injection layer, a hole-blocking layer, an emitter layer, an electron-blocking layer, an electron-transport layer, an electron-injection layer, an emitter layer, a charge-generation layer, a photon-absorption layer and/or an interlayer. It is preferably an emitter layer. The corresponding functional units in the layer can either be bonded to the polymer through one or more of the radicals R¹ to R⁹ in the general formula (I) being substituted by a corresponding group or the functional units can be present in the formulation in the form of a mixture with the polymer, so that they are distributed in the layer after application of the formulation and removal of the solvent, but are not covalently bonded to the polymer.

The present invention furthermore relates to an electronic device containing a polymer or blend, as defined above. As already stated above, it is preferred for the polymer to be present in a layer in the electronic device. Correspondingly, the layer can be a hole-transport layer, a hole-injection layer, a hole-blocking layer, an emitter layer, an electron-blocking layer, an electron-transport layer, an electron-injection layer, an emitter layer, a charge-generation layer, a photon-absorption layer and/or an interlayer, preferably an emitter layer.

The device may furthermore comprise layers built up from small molecules (SMOLED). These can be generated by evaporation of small molecules in a high vacuum.

The organic electroluminescent device comprises cathode, anode and at least one emitting layer. Apart from these layers, it may also comprise further layers, for example in each case one or more hole-injection layers, hole-transport layers, hole-blocking layers, electron-transport layers, electron-injection layers, exciton-blocking layers and/or charge-generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer). Interlayers, which have, for example, an exciton-blocking function, may likewise be introduced between two emitting layers. It should be pointed out, however, that each of these layers does not necessarily have to be present. These layers may comprise the polymers according to the invention, as defined above. It is also possible for a plurality of OLEDs to be arranged one above the other, which enables a further increase in efficiency with respect to the light yield to be achieved.

The electrodes (cathode, anode) are selected for the purposes of the present invention in such a way that their potential corresponds as well as possible to the potential of the adjacent organic layer in order to ensure the most efficient electron or hole injection possible.

The cathode preferably comprises metals having a low work function, metal alloys, metal complexes or multilayered structures comprising various metals, such as, for example, alkaline-earth metals, alkali metals, main-group metals or lanthanides (for example Ca, Ba, Mg, Al, In, Mg, Yb, Sm). In the case of multilayered structures, further metals which have a relatively high work function, such as, for example, Ag, can also be used in addition to the said metals, in which case combinations of the metals, such as, for example, Ca/Ag or Ba/Ag, are then generally used. It may also be preferred to introduce a thin interlayer of a material having a high dielectric constant between a metallic cathode and the organic semiconductor. Suitable for this purpose are, for example, alkali-metal or alkaline-earth metal fluorides, but also the corresponding oxides (for example LiF, Li₂O, BaF₂, MgO, NaF). The layer thickness of this layer is preferably between 1 and 10 nm.

The anode preferably comprises materials having a high work function. The anode preferably has a potential of greater than 4.5 eV vs. vacuum.

Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent in order to enable either irradiation of the organic material (O—SCs) or the coupling-out of light (OLEDs/PLEDs, O-LASERS). A preferred structure uses a transparent anode. Preferred anode materials here are conductive mixed metal oxides. Particular preference is given to indium tin oxide (ITO) or indium zinc oxide (IZO). Preference is furthermore given to conductive, doped organic materials, in particular conductive doped polymers, for example poly(ethylenedioxythiophene)/polystyrenesulfonic acid (PEDOT/PSS) or polyaniline (PANI).

The device is, in a manner known per se depending on the application, correspondingly structured, provided with contacts and finally hermetically sealed, since the lifetime of such devices is drastically shortened in the presence of water and/or air.

The invention is explained in greater detail below with reference to working examples, which, however, should not be regarded as restrictive of the scope of the invention.

### WORKING EXAMPLES

### Example 1

### Monomer Synthesis

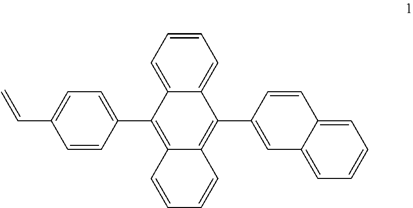

57.5 g (150 mmol) of 9-bromo-10-(2-naphthyl)anthracene, 25.0 g (169 mmol) of 4-vinylbenzeneboronic acid and 66.9 g of tripotassium phosphate are initially introduced. 400 ml of toluene, 100 ml of dioxane and 400 ml of water are then added. The reaction mixture is degassed for 10 minutes, and 1.37 g (4.50 mmol) of tri-o-tolylphosphine and 168 mg (0.75 mmol) of palladium acetate are added successively. After refluxing for 16 hours, the mixture is cooled to room temperature, the precipitated solid is filtered off with suction and washed with ethanol. After Soxleth extraction with toluene, the solid is washed again with ethanol, the mother liquor is evaporated to a slurry-like consistency, 500 ml of ethanol are then added, the precipitated solid is filtered off with suction and dried in vacuo. The yield is 20 g of compound 1 in a purity of 95%.

### Example 2

### Polymer Synthesis

The amounts indicated in Table 1 of monomer 1 (m₁) are dissolved in 200 ml of dry toluene, the corresponding amount of styrene (mₛₜ) is added (see Table 1), and 0.4 ml of sec BuLi (c=1.4 M) is added dropwise. A colour change from pale-yellow to brown takes place within 5 minutes. After 16 hours, the reaction is terminated by addition of methanol. The reaction mixture is evaporated to dryness in a rotary evaporator, and the residue remaining is taken up in tetrahydrofuran (THF). The polymer is precipitated by addition of methanol, filtered off with suction and dried in vacuo, giving 5 polymers P1 to P5, each with different monomer proportions (see Table 1).

TABLE 1

| | | | N₁ | N_{St} | γ = | |
|---|---|---|---|---|---|---|
| No. | m₁ [g] | m_{St} [g] | [mmol] | [mmol] | Nₛₜ/N₁ | Pₜₕₑₒ |
| P1 | 2.5 | 0.64 | 6.15 | 6.15 | 1.0 | 17 |
| P2 | 2.4 | 2.0 | 5.90 | 19.19 | 3.3 | 45 |
| P3 | 2.1 | 2.6 | 5.17 | 24.95 | 4.8 | 54 |
| P4 | 1.1 | 2.7 | 2.71 | 25.91 | 9.6 | 51 |
| P5 | 0.7 | 3.5 | 1.72 | 33.59 | 19.5 | 63 |

### Example 3

### Production of a PLED

The production of a polymeric organic light-emitting diode (PLED) has already been described many times in the literature (for example in WO 2004/037887 A2). In order to explain the present invention by way of example, a PLED is produced by spin coating with polymers P1 to P5 from Example 2 (with different proportions of the monomers). In order to obtain blue-emitting singlet emission, singlet emitter E1 is added to the solutions in a concentration of 5% by weight, based on the total weight of emitter and matrix.

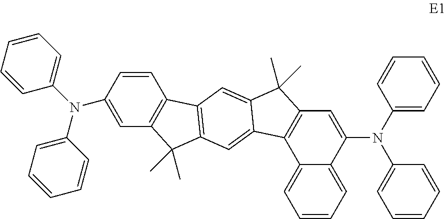

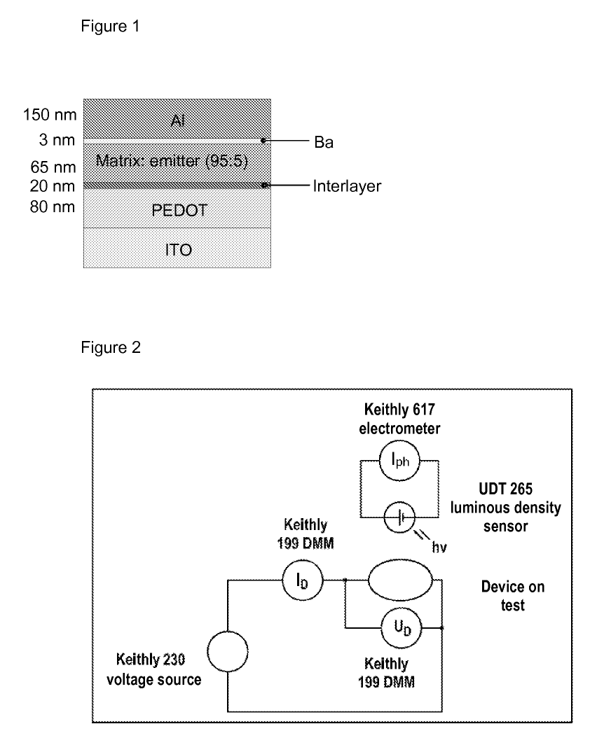

A typical device has the structure depicted in FIG. 1.

To this end, use is made of substrates from Technoprint (soda-lime glass) to which the ITO structure (indium tin oxide, a transparent, conductive anode) is applied.

The substrates are cleaned with deionised water and a detergent (Deconex 15 PF) in a clean room and then activated by UV/ozone plasma treatment. An 80 nm layer of PEDOT (PEDOT is a polythiophene derivative (Baytron P VAI 4083sp.) from H. C. Starck, Goslar, which is supplied as aqueous dispersion) is then applied as buffer layer by spin coating, likewise in a clean room. The spin rate required depends on the degree of dilution and the specific spin coater geometry (typical for 80 nm: 4500 rpm). In order to remove residual water from the layer, the substrates are dried by heating at 180° C. on a hotplate for 10 minutes. Then, firstly 20 nm of an interlayer (typically a hole-dominated polymer, here HIL-012 from Merck) and then 65 nm of the polymer layers are applied from toluene solutions (concentration of interlayer 5 g/l, for polymers P1 to P5 in each case 8 g/l and thus 0.42 g/l of E1) under an inert-gas atmosphere (nitrogen or argon). The two layers are dried by heating at 180° C. for at least 10 minutes. The Ba/Al cathode is then applied by vapour deposition (high-purity metals from Aldrich, particularly barium 99.99% (Order No. 474711); vapour-deposition units from Lesker, inter alia, typical vacuum level 5×10⁻⁶ mbar). In order to protect, in particular, the cathode against air and atmospheric moisture, the device is finally encapsulated and then characterised.

To this end, the devices are clamped in holders specifically manufactured for the substrate size and provided with spring contacts. A photodiode with eye response filter can be placed directly on the measurement holder in order to exclude influences from extraneous light. A typical measurement set-up is depicted in FIG. 2.

The voltages are typically increased from 0 to max. 20 V in 0.2 V steps and reduced again. For each measurement point, the current through the device and the photocurrent obtained is measured by the photodiode. In this way, the IVL data of the test devices are obtained. Important characteristic quantities are the maximum efficiency measured (“eff.” in cd/A) and the voltage U₁₀₀ required for 100 cd/m².

In order, in addition, to know the colour and the precise electroluminescence spectrum of the test devices, the voltage required for 100 cd/m² is applied again after the first measurement, and the photodiode is replaced by a spectrum measuring head. This is connected to a spectrometer (Ocean Optics) by an optical fibre. The colour coordinates (CIE: Commission International de l'éclairage, 1931 standard observer) can be derived from the measured spectrum.

The results obtained on use of polymers P1 to P5 in PLEDs are summarised in Table 2.

TABLE 2

| No. | n_{St}/n₁ | Pₜₕₑₒ | Mₙ (g/mol) | CIE (x/y) | Eff. (cd/A) | U₁₀₀ |
|---|---|---|---|---|---|---|
| P1 | 1.0 | 17 | 8100 | 0.14/0.13 | 0.99 | 12.1 |
| P2 | 3.3 | 45 | 9300 | 0.15/0.11 | 0.18 | 8.6 |
| P3 | 4.8 | 54 | 8500 | 0.16/0.12 | 0.22 | 10.3 |
| P4 | 9.6 | 51 | 7200 | 0.16/0.12 | 0.42 | 10.1 |
| P5 | 19.5 | 63 | 8100 | 0.15/0.07 | 0.23 | 10.9 |
| C1 | | | 9500 | 0.18/0.29 | 1.82 | 10.5 |

As can be seen from the results, polymers P1 to P5 represent a significant improvement over known non-conjugated and conjugated light-emitting polymers (e.g. C1) with respect to the colour coordinates. The novel polymers according to the invention are thus significantly more suitable for use in displays and lighting applications than polymers in accordance with the prior art.

1-12. (canceled)

13. A polymer, wherein said polymer comprises one or more optionally substituted styrene recurring units and one or more recurring units of formula (I) 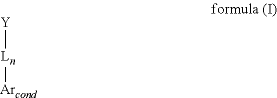
wherein
Y is a link to the polymer backbone;
Lₙ is an optionally substituted aromatic, heteroaromatic or non-aromatic group, or an alkylene, alkenylene or alkynylene group, where 3≧n≧1;
Ar_{cond} is a condensed, aromatic ring system having 10 to 40 C atoms or condensed, heteroaromatic ring system having 10 to 40 ring atoms, wherein at least one ring atom is a heteroatom, and the other atoms are C atoms, which is optionally substituted by one or more radicals R;
R is in each case, independently of one another, H, D, F, Cl, Br, I, N(R¹⁰)₂, N(Ar)₂, CR¹⁰═CR¹⁰Ar, Si(R¹⁰)₃, B(OR¹⁰)₂, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which is optionally substituted by one or more radicals R¹⁰, wherein one or more non-adjacent CH₂ groups is optionally replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NR¹⁰, O, S or CONR¹⁰ and wherein one or more H atoms is optionally replaced by F, Cl, Br, I, CN or NO₂, or an aryl or heteroaryl group having 5 to 60 ring atoms, which in each case is optionally substituted by one or more radicals R¹⁰, or an aromatic or heteroaromatic ring system having 5 to 40 aromatic ring atoms, which in each case is optionally substituted by one or more radicals R¹⁰, or an aryloxy or heteroaryloxy group having 5 to 40 aromatic ring atoms, which is optionally substituted by one or more radicals R¹⁰, or a combination of these systems; wherein two or more substituents R optionally define a mono- or polycyclic, aliphatic or aromatic ring system with one another, where at least one R is K—(Ar)ₘ, where m is greater than or equal to 1;
R¹⁰ is in each case, independently of one another, H, D or an aliphatic or aromatic hydrocarbon radical having 1 to 20 C atoms;
K is, in each case, independently of one another, a covalent bond, an optionally substituted aromatic, heteroaromatic or non-aromatic group, or an alkylene, alkenylene or alkynylene group; and
Ar is, on each occurrence, independently of one another, an optionally substituted aryl group, aryloxy group heteroaryl group, heteroaryloxy group, an aromatic or heteroaromatic ring system or a non-aromatic group.

14. The polymer of claim 13, wherein said polymer comprises one or more optionally substituted styrene recurring units and one or more recurring units of formula (Ia) 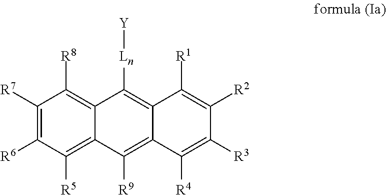
wherein
Lₙ is an optionally substituted aromatic, heteroaromatic or non-aromatic group, or an alkylene, alkenylene or alkynylene group, where 3≧n≧1;
R¹ to R⁹
are each, independently of one another, H, D, F, Cl, Br, I, N(R¹⁰)₂, N(Ar)₂, CR¹⁰═CR¹⁰Ar, Si(R¹⁰)₃, B(OR¹⁰)₂, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which is optionally substituted by one or more radicals R¹⁰, wherein one or more non-adjacent CH₂ groups is optionally replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NR¹⁰, O, S or CONR¹⁰ and where one or more H atoms is optionally replaced by F, Cl, Br, I, CN or NO₂, or an aryl or heteroaryl group having 5 to 60 ring atoms, which in each case is optionally substituted by one or more radicals R¹⁰, or an aromatic or heteroaromatic ring system having 5 to 40 aromatic ring atoms, which in each case is optionally substituted by one or more radicals R¹⁰, or an aryloxy or heteroaryloxy group having 5 to 40 aromatic ring atoms, which is optionally substituted by one or more radicals R¹⁰, or a combination of these systems; where two or more substituents R¹ to R⁹ may also form a mono- or polycyclic, aliphatic or aromatic ring system with one another, where at least one representative from R¹ to R⁹ is K—(Ar)ₘ, where m is greater than or equal to 1;
R¹⁰ is in each case, independently of one another, H, D or an aliphatic or aromatic hydrocarbon radical having 1 to 20 C atoms;
K is, in each case, independently of one another, a covalent bond, an optionally substituted aromatic, heteroaromatic or non-aromatic group, or an alkylene, alkenylene or alkynylene group;
Ar is, on each occurrence, independently of one another, an optionally substituted aryl group, aryloxy group heteroaryl group, heteroaryloxy group, an aromatic or heteroaromatic ring system or a non-aromatic group;
Y is a link to the polymer backbone.

15. The polymer of claim 13, wherein said polymer comprises units of formula (II), 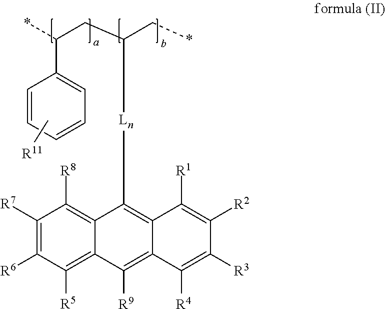
wherein
R¹¹ is H, D, F, Cl, Br, I, N(Ar)₂, CR¹²═CR¹²Ar, Si(R¹²)₃, B(OR¹²)₂, a straight-chain alkyl, alkoxy or thioalkoxy group having 1 to 40 C atoms or a branched or cyclic alkyl, alkoxy or thioalkoxy group having 3 to 40 C atoms, each of which is optionally substituted by one or more radicals R¹², wherein one or more non-adjacent CH₂ groups is optionally replaced by R¹²C═CR¹², C≡C, Si(R¹²)₂, Ge(R¹²)₂, Sn(R¹²)₂, C═O, C═S, C═Se, C═NR¹², P(═O)(R¹²), SO, SO₂, NR¹², O, S or CONR¹² and wherein one or more H atoms is optionally replaced by F, Cl, Br, I, CN or NO₂, or an aryl or heteroaryl group having 5 to 40 ring atoms, which in each case is optionally substituted by one or more radicals R¹², or an aromatic or heteroaromatic ring system having 5 to 40 aromatic ring atoms, which in each case is optionally substituted by one or more radicals R¹², or an aryloxy or heteroaryloxy group having 5 to 40 aromatic ring atoms, which is optionally substituted by one or more radicals R¹², or a combination of these systems; where two or more radicals R¹² optionally define a mono- or polycyclic aliphatic or aromatic ring system;
R¹² is, in each case, independently of one another, H or an aliphatic or aromatic hydrocarbon radical having 1 to 20 C atoms; and
a is a styrene-based recurring unit and b is a recurring unit of the general formula (I).

16. The polymer of claim 13, wherein said one or more of the radicals R¹ to R⁹ and/or R¹¹ each, independently of one another, are an electron-transport group, an electron-injection group, an electron-blocking group, a hole-transport group, a hole-injection group, a hole-blocking group, a photon-absorption group, an exciton-generating group and/or an emitter group.

17. A blend comprising the polymer of claim 13 and at least one further oligomeric, polymeric, dendrimeric or low-molecular-weight compound.

18. A formulation comprising the polymer of claim 13 in one or more solvents.

19. A compound of formula (III) 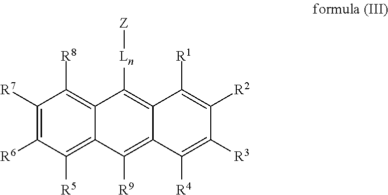
wherein
Lₙ is an optionally substituted aromatic, heteroaromatic or non-aromatic group, or an alkylene, alkenylene or alkynylene group, where 3≧n≧1;
R¹ to R⁹
are each, independently of one another, H, D, F, Cl, Br, I, N(R¹⁰)₂, N(Ar)₂, CR¹⁰═CR¹⁰Ar, Si(R¹⁰)₃, B(OR¹⁰)₂, a straight-chain alkyl, alkenyl, alkynyl, alkoxy or thioalkoxy group having 1 to 40 C atoms or a branched or cyclic alkyl, alkenyl, alkynyl, alkoxy or thioalkoxy group having 3 to 40 C atoms, each of which is optionally substituted by one or more radicals R¹⁰, where one or more non-adjacent CH₂ groups is optionally replaced by R¹⁰C═CR¹⁰, C≡C, Si(R¹⁰)₂, Ge(R¹⁰)₂, Sn(R¹⁰)₂, C═O, C═S, C═Se, C═NR¹⁰, P(═O)(R¹⁰), SO, SO₂, NO, O, S or CONR¹⁰ and where one or more H atoms is optionally replaced by F, Cl, Br, I, CN or NO₂, or an aryl or heteroaryl group having 5 to 60 ring atoms, which in each case is optionally substituted by one or more radicals R¹⁰, or an aromatic or heteroaromatic ring system having 5 to 40 aromatic ring atoms, which in each case is optionally substituted by one or more radicals R¹⁰, or an aryloxy or heteroaryloxy group having 5 to 40 aromatic ring atoms, which is optionally substituted by one or more radicals R¹⁰, or a combination of these systems; where two or more substituents R¹ to R⁹ also optionally define a mono- or polycyclic, aliphatic or aromatic ring system with one another, where at least one representative from R¹ to R⁹ is K—(Ar)ₘ, where m is greater than or equal to 1;
R¹⁰ is in each case, independently of one another, H, D or an aliphatic or aromatic hydrocarbon radical having 1 to 20 C atoms;
K is, in each case, independently of one another, a covalent bond, an optionally substituted aromatic, heteroaromatic or non-aromatic group, or an alkylene, alkenylene or alkynylene group;
Ar is, on each occurrence, independently of one another, an optionally substituted aryl group, aryloxy group heteroaryl group, heteroaryloxy group, an aromatic or heteroaromatic ring system or a non-aromatic group;
Z is a polymerisable group.

20. The compound of claim 19, wherein Z is selected from the group consisting of oxetane, epoxide, vinyl, vinyl ether, vinyl ester, and vinylamide.

21. An electronic device comprising the polymer of claim 13.

22. The electronic device of claim 21, wherein said electronic device comprises a hole-transport layer, a hole-injection layer, a hole-blocking layer, an emitter layer, an electron-blocking layer, an electron-transport layer, an electron-injection layer, an emitter layer, a charge-generation layer, a photon-absorption layer, and/or an interlayer.

23. The electronic device of claim 21, wherein said electronic device comprises a plurality of layers selected from hole-transport layer, hole-injection layer, hole-blocking layer, emitter layer, electron-blocking layer, electron-transport layer, electron-injection layer, emitter layer, charge-generation layer, photon-absorption layer, and/or interlayer.

24. The electronic device of claim 21, wherein said electronic device is an organic electroluminescent device/diode, an organic polymeric device/diode, an organic integrated circuit, an organic field-effect transistor, an organic thin-film transistor, an organic light-emitting transistor, an organic solar cell, an organic optical detector, an organic photoreceptor, an organic field-quench device, a light-emitting electrochemical cell, or an organic laser diode.
